(19) United States
(12) Patent Application Publication
(10) Pub. No.: US 2020/0204621 A1
(43) Pub. Date: Jun. 25, 2020

(54) MANAGEMENT OF LIVE MEDIA CONNECTIONS

(71) Applicant: York Telecom Corporation, Eatontown, NJ (US)

(72) Inventors: Luke Brown, Philadelphia, PA (US); Jennifer R. Schell, Colts Neck, NJ (US); John Mitchell Vitale, Mooresville, NC (US)

(21) Appl. No.: 16/230,271

(22) Filed: Dec. 21, 2018

(51) Int. Cl. H04L 29/08; H04L 29/06; G16H 80/00

(52) U.S. Cl. CPC H04L 67/1059; H04L 67/1074; H04L 65/4076; G16H 80/00; H04L 65/4084; H04L 65/60; H04L 65/1069

(57) ABSTRACT

A peer-to-peer media sharing network is facilitated by a central controller which maintains a map of media stream devices acting as sources, destinations, and relays for media streams. The controller maintains a remote connection with each media stream device, monitoring operational status and modifying the map as required in response to conditions. Each media stream device manages its own remote peer connections with other media stream devices by, e.g., initiating a peer connection once both the destination node and the desired stream are available. For simplicity and rapid response to changing status conditions, the map may include node groups comprising various types of nodes, such as aggregation groups, distribution groups, and link groups. Media stream devices may autonomously alter connections within groups as necessary to maintain connections.

### BACKGROUND

This disclosure pertains to peer-to-peer media sharing networks.

### SUMMARY

A network for sharing live media streams comprises a controller and multiple nodes. Each node that transmits and/or receives media content has a unique identity in the system. Each node is connected to a controller which provides coordination for the system. The controller may be, for example, a cluster of servers. A node may have one or more devices attached to the node that capture media streams, such as audio and video content. Nodes establish peer connections with other nodes, over which they transmit streams. Each peer connection may carry multiple streams of media content in each direction.

A node may receive two types of inbound streams: local and remote. A local inbound stream is the media captured by devices physically attached to it. Remote inbound streams are those it receives from its peer connections. Each stream is identified by the media node from which it originates. The inbound streams are available to be sent to output devices connected to the node, such as audio and video output devices. A node may transmit its local and remote inbound streams to other nodes over peer connections. The media captured by a node may be relayed multiple times across nodes.

Peer connections may be established automatically on demand. When a first node initiates the transmission of a stream to a second node with which it does not already have a peer connection, the first and second nodes may establish a new peer connection without any user involvement.

At a given time, a peer connection may be interpreted as being in one of multiple states, e.g. on, off, turning on, and turning off. For example, during automatic establishment of a new peer connection, the connection may be in a “turning on” state. Once the peer connection is established, it may be considered to be in the “on” state. In the case of a communications failure, or if the controller instructs the node to terminate a peer connection, then the peer connection may be deemed in the “turning off” state, and the nodes involved will begin to cleanly terminate the connection. Once a peer connection is fully closed, the connection is effectively nonexistent and interpreted to be in the “off ” state.

The intermediate “turning on” and “turning off ” states allow nodes to handle concurrent signals as the nodes begin turning on or off, ensuring the peer connections quickly and efficiently transition to either “on” or “off”. The nodes may inform the controller of each peer state transition, allowing the controller to maintain the state of each connection between nodes.

Nodes may detect stream terminations and respond to those terminations immediately, resulting in a cascade of terminations through the network of relayed streams. When a peer connection turns off, a node may remove any inbound remote streams it was receiving from that connection. If the node had been relaying any of those inbound remote streams to other nodes, the node may remove those outbound streams from those other peer connections. Likewise, if an individual local or remote inbound stream terminates for any reason, the node may remove that stream from any outbound peer connections. Stream terminations may cascade through outbound connections as many times as they have been relayed onward.

Each node may maintain a connection state with the controller of being either online or offline. If a node's connection with the controller goes offline while it continues to maintain active peer connections, those peer connections may remain active during a grace period configured by the controller. The node may make repeated attempts to reconnect with the controller while its connection is offline. If the grace period expires before the node's connection with the controller comes back online, the controller may instruct other nodes to terminate peer connections with the offline node. The grace period serves to protect the network of peer connections from brief network outages that do not otherwise affect the streams, as well as from temporary outages of the controller itself.

A node may receive streams passively. A node may track the originating node of each remote stream it receives from each of its peers. A node may also track the availability of local streams, for example, according to the state of its local stream acquisition devices. Each node may thus maintain a set of active inbound streams, and automatically add new streams to the set as the new streams become available. Similarly a node may automatically remove streams from the set as they become unavailable.

The controller may inform a node which streams the node should transmit, and where to transmit each stream. This information may include the expected state of peer connections among the media device nodes in the streaming network. The transmitting node may then use this information to attempt to establish or use the peer connections. If either the stream or the destination node is not available, then the transmitting node may wait for the availability of both the stream and the destination node before attempting to initiate streaming.

The controller may similarly inform a transmitting node to stop transmitting a stream. This information may instruct the transmitting node to stop transmitting generally, stop transmitting a particular stream, and/or stop transmitting to specified destination nodes. The transmitting node may then remove a corresponding stream transmission entry from its set of expected outbound transmissions, and terminate any such stream if the stream is active.

The controller may manage all connections, such that the stream termination node themselves do not alter the set of expected outbound transmissions. Rather, the controller may centrally coordinate all connections, and all the content expected to be delivered on the connections. The nodes which create and consume the streams may then act as commanded by the controller.

The network may support operations such as remote observation of individuals, groups, facilities, or equipment. For example, the system may support a network of nodes creating streams of medical patients and nodes displaying streams of the patients to clinical observers, wherein no two-way conferencing link exists between any two endpoints. Rather, the controller coordinates a network of endpoints for sourcing and sinking one-way streams of information, with the streams flowing directly through the nodes to one another, without the streams traversing the controller.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to limitations that solve any or all disadvantages noted in any part of this disclosure.

### BRIEF DESCRIPTION OF THE FIGURES

A more detailed understanding may be had from the following description, given by way of example in conjunction with the accompanying drawings.

FIG. 1 is a block diagram of an example network.

FIG. 2 is a call flow of an example of initiating the relaying of a stream.

FIG. 3 is a call flow of an example of configuring a streaming network.

FIGS. 4-19 are block diagrams of example network configurations for nine example deployments, and the associated streams.

FIG. 4 is a network block diagram of a first example deployment, with groups for broadcast nodes and observation nodes with remote connectivity.

FIG. 5 illustrates streams for the network of the example of FIG. 4.

FIG. 6 is a network block diagram of a second first example deployment, with groups for broadcast nodes on a shared network and remote observation nodes.

FIG. 7 illustrates streams for the network of the example of FIG. 6.

FIG. 8 is a network block diagram of a third example deployment, with groups for broadcast nodes and observation nodes on a shared network.

FIG. 9 illustrates streams for the network of the example of FIG. 8.

FIG. 10 is a network block diagram of a fourth example deployment, with groups for wide-distribution surveillance application on a shared network.

FIG. 11 illustrates streams for the network of the example of FIG. 10.

FIG. 12 is network block diagram of a fifth example deployment, with groups for a large-scale surveillance application.

FIG. 13 illustrates streams for the network of the example of FIG. 12.

FIG. 14 is network block diagram of a sixth example deployment, with failures in a large-scale surveillance application.

FIG. 15 illustrates updated streams for the network of the example of FIG. 14, in response to the failures.

FIG. 16 is network block diagram of a seventh example deployment, with of groups for extended large-scale surveillance application.

FIG. 17 is network block diagram of an eighth example deployment, with groups for a general conference application.

FIG. 18 illustrates streams for the network of the example of FIG. 17.

FIG. 19 is network block diagram of an eighth example deployment, with groups for a large-scale conference application.

### DETAILED DESCRIPTION

Networks of devices for media stream broadcast, relay, and observation may be organized and maintained by a central controller, e.g., through organization into hierarchical structures, whereby media is streamed peer-to-peer, e.g., without passing through the controller, to exploit diverse hardware resources and networking topologies.

Such networks may be built, for example, via extensions to existing media protocols such as WebRTC. WebRTC is a commonly used software component that provides capabilities for the capture and transmission of audio and video streams. Traditionally, WebRTC implementations coordinate stream transmissions between machines via peer-to-peer “signaling” which is separate from the media streams themselves. A common standard for this signaling is the Session Initiation Protocol (SIP), which was originally designed to implement telephone service.

However, in the context of telecommunication services for the exchange of live media streams, SIP introduces unnecessary complexities and burdens. For example, SIP includes legacy telephone service features, such as dialing, ringing, holding, and transferring calls, which are often add unnecessary complexity for services that do not directly use those features.

Normally, for example, WebRTC uses an offer/answer protocol and a separate signaling channel. The initiating node creates an offer message, sends it to the other node through the controller, and the other node establishes its side of the peer connection and responds automatically with an answer message through the controller. When a node initiates the transmission of a stream to another node with which it already has an active peer connection, it adds the transmission to that existing connection.

While the use of peer-to-peer media distribution is desirable, in the design infrastructure for large-scale audio/video services, such as video teleconferencing, broadcasting, or surveillance, traditional telephony signaling may not be appropriate. Telephony signaling assumes that connections, like phone calls, are temporary, and occur from point to point between various changing parties. The assumed workflow is that a connection is established when one party dials one recipient who answers, and that the connection continues until either party ends the call or an infrastructure failure drops the call. Therefore, resuming a connection after it is dropped, for example, requires that one of the parties starts the process over by dialing the other party.

In contrast, for media streaming services, such as live video conference, broadcast, and surveillance, it is often advantageous for connections to be restored immediately upon the return of available resources, without the intervention of a user or the renegotiation of connections. Rather, it is beneficial for connections to be re-established, the moment an infrastructure failure is resolved, e.g., at the moment power is returned to a device.

Thus, traditional WebRTC implementations, such as web browsers, have practical limitations that make them unsuitable for implementing a large-scale infrastructure on their own. A system design that orchestrates the cumulative aggregation and distribution of streams, while leveraging some features of traditional peer-to-peer implementations such as WebRTC, may overcome these limitations for certain large-scale telecommunication infrastructure requirements.

Similarly, the infrastructure of a large-scale audio/video service may be tailored to its expected usage patterns and scale. The type of application, such as conferencing, broadcasting, or surveillance, may be considered in determining the degree to which participating machines are interconnected. The scale of the infrastructure may then correspond, e.g., to the peak capacity of machines sending and receiving interconnected audio/video streams. However, instead of building an infrastructure with software components that are purpose-built for a specific type of application and expected peak usage, an adaptable infrastructure may be implemented with interchangeable general-purpose end nodes and relay entities that are dynamically assembled and grouped to perform the desired audio/video application at the desired scale at a particular time.

### Illustration of General Concept

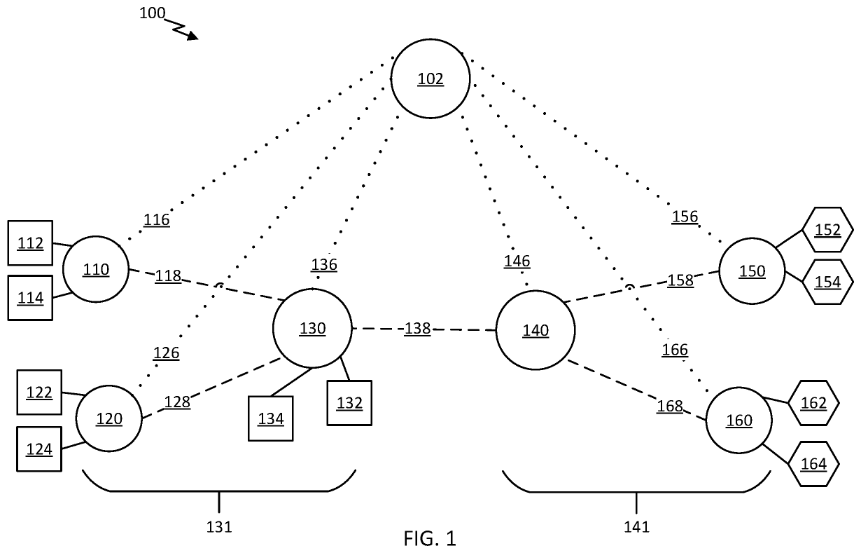

FIG. 1 is a block diagram of an example media sharing system 100 comprising multiple nodes, comprising: a controller 102; media nodes 110, 120, 130, 140, 150, and 160; media input devices 112, 114, 122, 124, 132, and 134; and media output devices 152, 154, 162, and 164. Each media node 110, 120, 130, 140, 150, and 160 is connected to the controller 102 and has a unique identifier within the system. The media nodes 110, 120, 130, 140, 150, and 160 nodes and the controller 102 each include one or more computing devices including a processor and memory for management of network operations, for example, and/or the processing of media streams and other data. The controller 102 may be, for example, a server or a cluster of servers that provide coordination for the system 100. In practice system 100 may contain any number of nodes, depending on the capacity of the controller 102 and the requirements of particular applications.

In addition to communicating with the controller 102, each media node 110, 120, 130, 140, 150, and 160 may be capable of inputting and outputting media streams locally, as well as transmitting and receiving media streams over one or more network connections formed with other media nodes. In general, the controller 102 does not receive or transmit media streams.

The controller 102 maintains a graph (or map) of a desired organization of the connections and streams flowing in the system. The controller 102 informs each of the media nodes of the desired operations of that media node. The controller 102 further monitors the status of each media node 110, 120, 130, 140, 150, and 160, and the peer connections among these media nodes, and may adjust the graph of the connections and streams accordingly.

In the example of FIG. 1, the media nodes are arranged in two groups. Group 131, comprising nodes 110, 120, and 130, is an aggregation node group. An aggregation node group is a hierarchy in which the inbound steams of a number of media nodes at a lower level in the hierarchy are available at a node at a higher level in the hierarchy. Here, nodes 110 and 120 are the lower level nodes which feed into the higher level node 130. Here, only two levels of hierarchy are depicted. In practice, the hierarchy may extend to any number of levels, for example, with streams fanning in from an increasing number of nodes at lower levels.

Any media node may receive local inbound streams from media source input devices attached to the media node that capture audio, video, or other media content. In the example of FIG. 1, media node 110 receives local inbound streams from media devices 112 and 114, to which the node 110 is physically attached. Media devices 112 and 114 may be a camera and a microphone, for example. A node such as node 110 may additionally or alternatively be locally connected to other local stream source devices, such as sensors, telemetry equipment, and repositories of recorded media, for example. Herein the terms “media” and “stream” generally refer to any stream data, such as audio, video, telemetry, sensor, recordings, or the like, and include, but are not limited to, one-way and two-way teleconferencing and surveillance.

Herein the term “physically attached” refers generally to means by which computing devices may be attached to a local peripheral device, as by wire, fiber-optics, a local area network, or infra-red beam, short-range radio connections, and the like.

Node 110 has a remote connection 116 with the controller 102. Via connection 116, node 110 receives instructions from the controller 102. Herein the term “remote connection” refers generally to means by which computing devices may be connected to each other at some distance, such as via Internet protocol packet switched networks, cellular connections, and the like.

Node 110 has a remote connection 118 with a node 130. Remote connection 118 is a peer connection 118 which allows node 110 to transmit and receive remote streams to and from node 130. Each peer connection may carry multiple streams of media content in either direction, or in both directions. The content of each stream carried over a peer connection is identified by the media node that captured the stream, and may be further identified by the nodes by which it is relayed, if any. For example, node 110 may combine the inputs from the input devices 112 and 114 into a single stream, which node 110 labels as stream A of node 110, and sends to node 130.

In the example of FIG. 1, node 120 also has two locally attached media source devices 122, and 124, which may be patient monitoring devices, for example. Node 120 is connected to the controller via a remote connection 126. Node 120 has a remote peer connection 128 with node 130.

Node 130, which is the top of the aggregation group 131, has a remote connection 135 with the controller 102, and peer connections 118, 128, and 138 with node 110, node 120, and node 140, respectively.

Node 130 further has inbound local media streams from locally attached media source devices 132 and 134, which may an audio player of pre-recorded announcements and a video recorder, for example.

All of the inbound streams of aggregation group 131 are available to nodes having peer connections with node 130. The inbound streams include those from local inputs 132 and 134, from node 110 via connection 118, and from node 120 via connection 128. Node 140 has a peer connection with node 130, and therefore may receive any of the streams in aggregation group 131. Thus, node 140 may receive streams from the devices 132 and 134 which are attached to node 130. Node 140 may further receive streams from nodes 110 and 120 as relayed by node 130. Thus node 140 may receive streams from devices 112, 114, 122, and 124.

The second node group in the example of FIG. 1 is a distribution node group 141, comprising nodes 140, 150, and 160. A distribution node group is a hierarchy in which the inbound steams of a single node at a higher level in the hierarchy are available to a number of nodes at a lower level in the hierarchy. Here, node 140 is the higher level node which feeds streams into lower level nodes 150 and 160. Here, only two levels of hierarchy are depicted. In practice, the hierarchy may extend to any number of levels, for example, with streams fanning out to an increasing number of nodes at lower levels.

Node 140 is the top node in the hierarchical distribution node group 141. Node 140 has a remote connection with the controller 102, and remote peer connections 138, 158, and 168 with nodes 130, 150, and 160, respectively. Node 140 receives inbound streams from node 130, and feeds those streams to nodes 150 and 160.

Node 150 is connected to the controller via connection 156, and has a remote peer connection 158 with Node 140. Node 140 receives remote inbound streams node 140. Any node may send received streams to one or more local media output devices. Node 150 send streams received inbound from node 130 to local to media output devices 152 and 154. Output devices 152 and 154 are physically attached to node 150.

Similarly, node 160 has a remote connection 166 with the controller 102, and locally attached media output devices 162 and 164. Node 160 has a remote peer connection 168 with the node 140.

The controller 102 may present the intended connections to the nodes in a variety of ways, and the media nodes may optionally exercise various levels of autonomy in implementing an intended organization communicated by the controller 102. Table 1 illustrates an example graph of intended connections where all the nodes of FIG. 1 are treated as members of a simple group. In other words, in the example of Table 1, the controller 102 does not treat the nodes as belonging to an aggregation node group or distribution node group. Rather, each stream is identified by its endpoints and relays. The endpoints are the media nodes which source (originate) and sink (receive) a stream transmission. Relays are media nodes with receive streams and both them along to other nodes.

TABLE 1

Endpoints

| Source | Sink | Relays |
|---|---|---|
| 110 | 150 | 130, 140 |
| 120 | 150 | 130, 140 |
| 130 | 160 | 140 |

Table 2 illustrates an alternative way of organizing nodes using an aggregation node group and a distribution node group. The values of Table 2 correspondent to the arrangement depicted in the example of FIG. 1.

TABLE 2

| Group | Group Type | Node | Node Type |
|---|---|---|---|
| 131 | Aggregation | 110 | lower |
| | | 120 | lower |
| | | 130 | top |
| 141 | Distribution | 140 | top |
| | | 150 | lower |
| | | 160 | lower |

Table 3 illustrates an example of further data that may be presented to the nodes, along with Table 2, to enable the media nodes to establish a needed peer connections.

TABLE 3
Endpoints

| Source | Sink |
|---|---|
| 110 | 150 |
| 120 | 150 |
| 130 | 160 |

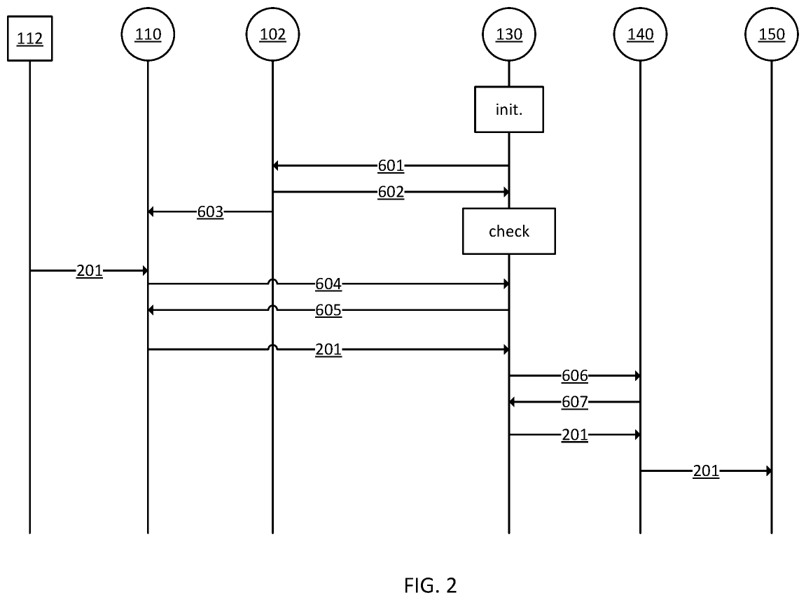

FIG. 2 is a call flow of an example process for managing the exchange of media streams. For purposes of illustration, the example of FIG. 2 uses the devices and connections of FIG. 1. In the example of FIG. 2, it is assumed that all nodes are online with the controller 102, and all peer connections are in the “on” state. It is further assumed that all nodes belong to a single simple group of nodes, rather than being divided, for example, into an aggregation node group and a distribution node group, e.g., described in reference to Table 1.

As with all methods described herein, it will be appreciated that example method of FIG. 2 may be applied in a variety of network topologies.

In the first step of FIG. 2, media node 130 reboots. Node 130 then informs the controller 102 in message 601 that node is 130 is operational. The controller 102 responds to node 130 by providing information in a message 602 about which streams to node 130 should receive and where to send them. Message 602 indicates that a stream from media node 110 should be relayed to node 140 when it becomes available. Optionally, in message 603, the controller 102 informs media node 110 that media node 130 is now online.

Next, media node 130 checks its status, and seeing that it is currently receiving no streams, merely waits.

At some time later, an input device 112 begins to provide media node 110 with a media stream 201. Media node 110 has been previously configured by the controller 102 to send such a stream to node 130. Once the stream is available, node 110 contacts node 130 in a message 604 to initiate a peer connection. Since peer connections can be costly to maintain in terms of device and network resources, nodes 110 and 130 do not establish a peer connection until the stream is available and the connection is possible. In message 605, node 130 confirms the establishment of the peer connection, and node 110 begins to send stream 201 to node 130.

Once the stream 201 is available, node 130 sends a message 606 to node 140 to initiate establishment of a peer connection between node 130 and node 140. In this example, node 140 has already been configured by the controller 102 to accept stream 201 from node 130. Node 140 responds with a message 607 to confirm establishment of a peer connection, and node 130 begins to send stream 201 to node 140.

If needed, node 140 may then establish a peer connection with node 150. In this example, a connection is already established, and node 140 immediately begin to send stream 201 to node 150.

In the example of FIG. 2, the controller 102 is in communication with nodes 110, 130, 140, and 150 to monitor the online and connection status of the system, and to provide configuration data to each of nodes 110, 130, 140, and 150. However, no media data is passed through controller 102. Nor does controller 102 take any direct part in the establishment of peer connections among media nodes, nor in the initiation of streams. Rather, the media nodes 110, 130, 140, and 150 independently manage their own connections and streams in accordance with the configuration provided by the controller 102.

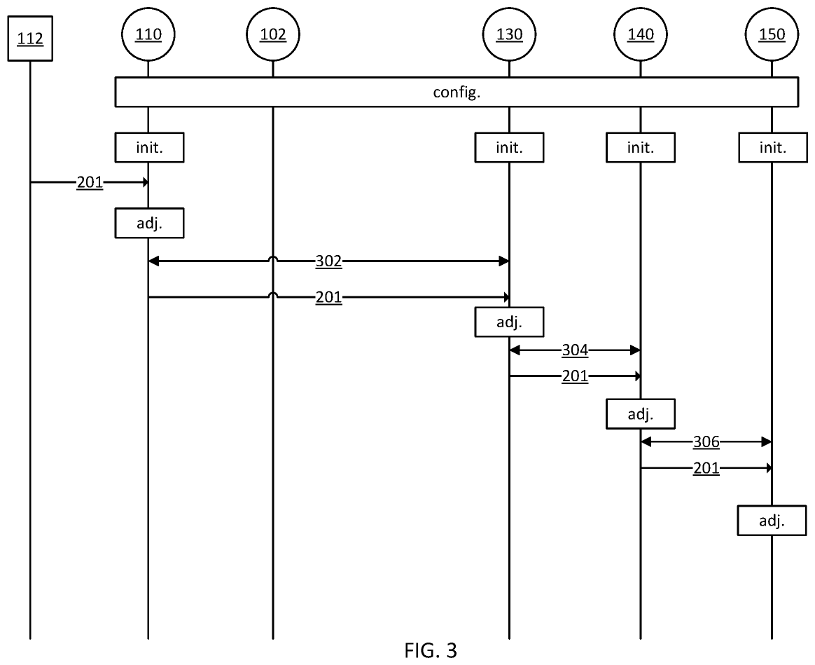

FIG. 3 is a call flow of another example process managing the exchange of media streams. For purposes of illustration, the example of FIG. 2 uses the devices and connections of FIG. 1. In the example of FIG. 3, it is assumed that all nodes are online with the controller 102 and all peer connections are in the “off ” state. It is further assumed that nodes 110 and 130 belong to an aggregation node group, and that nodes 140 and 150 belong to a distribution node group, as was described in reference to FIG. 1.

In the example of FIG. 3, the intended configuration is sent by the controller to the nodes 110, 130, 140, and 150. This may be done, for example, by the controller sending individual messages to each of the nodes individually or in a broadcast to all nodes. Alternatively, the controller 102 may send the intended configuration in individual messages, or a broadcast, to the top nodes in the aggregation and distribution node groups, nodes 130 and 140 respectively, whereby the top nodes 130 and 140 propagate the configuration to their respective lower nodes 110 and 150. In practice, such configuration information may include status information regarding which nodes are online with the controller, for example, and which peer connections are in the on, off, turning off, and turning on states.

Upon receiving the configuration information, the nodes 110, 130, 140, and 150 initialize tables of which streams are meant to transmitted, from which sources, and to which destinations. In the example of FIG. 3, all of the nodes are online, but none of the peer connections are on. The nodes 110, 130, 140, and 150 are configured to process streams whenever the necessary streams and connections are available. To conserve node resources, peer connections will not be established until streams are available.

The configuration stipulates that a stream 201 from node 110 is to be relayed via nodes 130 and 140 to node 150. Initially, stream 201 is not available. Therefore, upon receiving the configuration information, the nodes do not immediately endeavor to form the necessary connections.

At some point, a stream source device 112, e.g., a video camera with a microphone, which has a local, physical connection to node 110, begins send a stream 201 to node 110. Node 110 then adjusts an internal list of available streams by noting the availability of the stream 201 from stream source 112. Node 110 may then compare this list to the configuration provided by the controller 102 to determine where the stream is to be sent. In step 302, node 110 initiates and establishes a peer connected with node 130. Once the connection is established, node 110 begins sending stream 201 to node 130.

The automatic connection of peer media stream devices continues. Once node 130 begins to receive stream 201, node 130 adjusts an internal set of available streams, and compares this to the configuration it received from the controller 102. In step 304, node 130 establishes the necessary peer connection with node 140. Node 130 then begins transmission of stream 201 to node 140.

Similarly, node 140 then adjusts an internal list of available streams, and compares this to the configuration provided by controller 102. In step 306, node 140 establishes the necessary connection with node 150, and next begins to send stream 201 to node 150. Node 150 then updates its internal list of available streams, etc.

The cascading of adjustments to conditions and management of peer connections occurs without the direct intervention of the controller. It may even occur while the controller is offline. Rather, the media stream device nodes, once configured by the controller, act independently to acquire and relay streams as the streams and connections become available.

Streams from Multiple Sources and to Multiple Destinations

In the example of FIG. 3, a single stream follows a single path to a single destination, via two relaying nodes. In practice, the stream could follow a variety of paths. The path used may be chosen opportunistically by the media stream devices themselves dynamically in response to network conditions. For example, if there were more than one aggregating group top nodes, where node 110 sent stream to both top nodes, and the distribution top node 140 were configured to accept stream 201 from either top node, then node 140 would form a connection with whichever aggregation top node came online first. Indeed, the configuration distribution group to which node 140 belongs may stipulate that stream 201 may originate from any top node of the aggregation group, e.g., by specifying the group number from which the stream is to arrive, rather than a specific node which is expected to provide the stream. Through similar mechanisms, networks of media stream devices may detect, adjust to, and compensate for the loss or addition of various nodes and pathways.

### Reuse of Connections

When a node initiates transmission of a stream to another node with which it already has an active peer connection, it may simply add the transmission to that existing connection.

### Connection States

The controller may instruct a node to terminate a peer connection. The node then transitions the peer connection to the “turning off ” state and begins to cleanly terminate the connection. Once a peer connection is fully closed, the connection is effectively nonexistent and interpreted to be in the “off ” state. The intermediate “turning on” and “turning off ” states allow nodes to handle concurrent signals to begin turning on or off, ensuring the peer connections quickly and efficiently transition to either “on” or “off”. The nodes may inform the controller of each peer state transition, allowing the controller to maintain the state of each connection between nodes.

A node may detect stream terminations and respond to those terminations immediately, causing terminations to cascade through the network of relayed streams. When a peer connection turns off, a node may remove any inbound remote streams it was receiving from that connection. If the node had been relaying any of those inbound remote streams to other nodes, the node may remove those outbound streams from those other peer connections. Likewise, if an individual local or remote inbound stream terminates for any reason, the node may remove that stream from any outbound peer connections. Stream terminations cascade through outbound connections as many times as they have been relayed onward.

### Grace Periods

Each node maintains its connection state with the controller, e.g., as either online or offline. If the connection of a node to the controller goes offline while it continues to maintain active peer connections, those peer connections may remain up during a grace period configured by the controller, whereby the node makes repeated reconnection attempts to reconnect with the controller while its connection is offline. If the grace period expires before the connection with the controller goes back online, the controller may instruct peers of the node to terminate those peer connections. Such a grace period serves to protect the network of peer connections from brief network outages that do not otherwise affect the streams, as well as from temporary outages of the controller itself.

### Communicating System Status and Configuration

The controller may inform a node which streams the node should transmit, and where to transmit each stream. This information may include the expected state of the streaming network. The transmitting node may then use this information to attempt to establish connections. If either the stream or the destination node is not available, then the transmitting node may wait for the availability of both the stream and the destination node before attempting to initiate the stream.

The controller may similarly inform a transmitting node to stop transmitting a stream. This information may instruct the transmitting node to stop transmitting generally, stop transmitting a particular stream, and/or stop transmitting to specified destination nodes. The transmitting node may then remove a corresponding stream transmission entry from its set of expected outbound transmissions, and terminate any such stream if the stream is active.

A node may automatically remove streams from the set as they become unavailable. However, stream terminations themselves do not alter the set of expected outbound transmissions. Unless the map is altered by a command from the controller peer nodes will continue to attempt requested connections and transmissions with available resources. This includes automatically adding new streams as the new streams become available.

### Node Groups

Adding a peer connection to a node may incur greater resource demands on the physical machine than does adding a stream to a peer connection. Therefore, it may be beneficial for streams to be relayed in hierarchical patterns that accumulate capacity at the cost of increased latency per relay. For example, one pattern may be used for aggregated stream sourcing, and another pattern may be used for aggregated stream distribution.

For example, each of the nodes passing media content (e.g., broadcast nodes which originate streams in the network, relay nodes which process and transfer streams, and observation nodes which display media to human users) may belong to a node group. A given physical apparatus may host a variety of “nodes” for these purposes. For example, a first apparatus may be the source of a first media stream, a relay for a second media stream, and consumer of a third stream.

Node groups may be configured by a central controller, which maintains a map of all the nodes, their groupings, and the streams they should carry. The controller, for example, may group nodes into simple groups, aggregation groups, distribution groups, and interconnection groups. A controller may configure any number of node groups of each type.

The controller may maintain sets of media endpoints, where each set is a pair of nodes, with one node being the original source of a stream, and the other node is the destination of a stream. The destination may be a final destination or a relay point. The controller may track the media endpoints for each node group, such that the interpretation of the endpoint pairs corresponds to the group type, e.g., aggregation or distribution node group.

### Simple Node Groups

Nodes may be assigned to a simple node group when they do not relay streams with each other. A member of a simple node group may receive relayed streams only from nodes that are not in the same group. The controller identifies a media endpoint for each member of a simple node group, where the media source and the group member are the same.

### Aggregation Node Groups

An aggregation node group organizes nodes into a relay hierarchy, which can be defined as a vertical orientation, whereby each node forwards all of its inbound streams to one node at the next higher level. All inbound streams in the hierarchy are available from nodes at the top level. Members of an aggregation node group may have inbound local streams, whereby devices connected to the member nodes provide streams, which the member nodes share with the network. An inbound remote stream at the bottom level of an aggregation group may originate from another node group.

The hierarchical structure of an aggregation group may be configured with a sequence of numbers, for example, where the first number specifies the count of top-level nodes, and the rest of the sequence specifies per level how many nodes are to relay streams to a node at the next higher level. When each node is assigned to the group, the controller assigns it to a location within the configured hierarchy, e.g., beginning at the top level and proceeding downward, completing each successive level before continuing to subsequent levels. Assignments may be made within a level by rotation through the set of nodes at the next higher level, such that the number of nodes forwarding to each node at the same level differs by at most one. Once the configured hierarchy is fully assigned, any additional online nodes in the group may be designated as reserve nodes, which do not immediately participate in the hierarchy.

If a node assigned to the hierarchy goes offline, its assignment is dropped, and if a reserve node is available, then the reserve node is assigned to the newly vacant location in the hierarchy. If an assigned node goes offline and no reserve node is available, all assignments in the group may be reset. An alternative to resetting the group assignment is to preserve otherwise unaffected forwarded streams, but this may result in additional reassignment complexity and group management overhead.

The controller may identify a media endpoint for each media source available within an aggregation node group, and select the highest-level member node available for each media source. It may be advantageous that, at most, one endpoint is identified for each media source available within an aggregation node group.

### Distribution Node Groups

A distribution node group may be used to organize nodes into a relay hierarchy with a vertical orientation that is opposite that of an aggregation node group. Members of a distribution node group are expected to have no inbound local streams. An inbound remote stream at the bottom level of a distribution group originates from another node group. A node assigned to a level higher than the bottom receives all of its inbound remote streams from one node at the next lower level.

The hierarchical structure of a distribution node group may be configured with a sequence of numbers, where the first number specifies the count of bottom-level nodes, and the rest of the sequence specifies per level how many nodes at various levels should receive streams from a node at the next lower level. The controller may assign online nodes to the hierarchy as described for aggregation groups, but in the reverse vertical orientation, beginning at the bottom level and continuing upward. Likewise, once the configured hierarchy is fully assigned, any additional online nodes in the group may be designated as reserve nodes. If an assigned node goes offline, the controller may respond as described for aggregation node groups.

The controller may identify a media endpoint for each inbound stream among all nodes within the distribution group that do not further relay streams within the group. Unlike simple node groups and aggregation node groups, a distribution node group may identify many media endpoints for a given media source.

### Interconnecting Node Groups—Links and Media Propagation

The controller may be configured to link node groups together by maintaining a set of links where each link connects two node groups. Such links may be directional, where one node group is the source and the other is the destination. A node group may have links in either direction with any number of other node groups. A link may optionally specify a set of media sources that should be propagated from the source group to the destination group. A link may specify the propagation of all media sources available from the source group to the destination group.

For media propagation between groups, the controller may identify media endpoints from the source group that should be relayed to the destination group. If the source group is a simple node group or an aggregation node group, then the controller may select the distinct media endpoints for the media sources to be propagated. If the source group is a distribution node group, then the controller may select one media endpoint for each media source to be propagated, evenly distributing across nodes.

When the destination is a simple node group and media propagation is specified, the controller may forward each propagating media endpoint to every member of the simple node group.

When the destination is an aggregation node group, for each propagating media endpoint from the source group, the controller may identify one member of the destination group to which it should forward that stream. The destination nodes in the aggregation node group may be evenly distributed across all nodes in the bottom level of its hierarchy, for example.

Similarly, when the destination is a distribution node group, the controller may forward each propagating media endpoint from the source group to all nodes in the bottom level of the destination group hierarchy.

As the set of media endpoints from a group changes, the endpoint selection may be updated for each node group to which it is configured for media propagation.

### Interconnecting Node Groups—Direct Media Selection

The controller may inform an application of the available node groups and the media sources available from each group. An application may request that the controller select a media source from a node group to be forwarded to a specific destination node. The destination node is expected to be in a group that is linked to the source group, although the link need not be configured for media propagation.

The controller may respond to a direct media selection by identifying a corresponding media endpoint from the source group, if available, using the same procedure as is used for media propagation. The controller may forwards that media endpoint directly to the destination node.

As with media propagation, as the set of media endpoints from a group changes, the controller updates the media endpoints chosen for its outbound direct media selections.

### Interconnecting Node Groups—TURN Servers

In practice, network firewalls may block the direct establishment of a peer connection between nodes. To address this, systems such as WebRTC rely on technologies such as TURN servers to establish connectivity across firewalls. In the solutions described herein, in addition to forming simple, aggregation, and/or distribution groups, the controller may configure TURN server groups as interconnection groups, e.g., wherein each TURN server is assigned to one TURN server group. The controller may track its connectivity with each TURN server, monitoring its status as online or offline.

A TURN server group may be assigned to one or more links between node groups, whereby the controller informs each node of the set of individual online TURN servers to use when connecting to another given node. The controller may select all online TURN servers among the TURN server groups assigned to the links between the node groups, for example. The controller may alternatively provide a subset selection in rotation. A TURN server group with multiple members may enables connectivity between linked node groups in order to tolerate individual TURN server failures.

In addition to enabling connectivity across network firewalls, TURN servers may effectively aggregate streams. A TURN server functions as a proxy for peer connections between remote nodes. A node with multiple peer connections that traverse the same TURN server may entail fewer resource demands on the node than would the equivalent direct peer connections between individual nodes. Therefore, node group links with TURN server groups may be deployed as stream aggregators between node groups. A TURN server group aggregation may be enabled by configuring the group with fewer members than its connecting node groups. A TURN server group configuration may further enable this effective aggregation with a provision for subset selection in rotation.

### Example Deployment Configurations

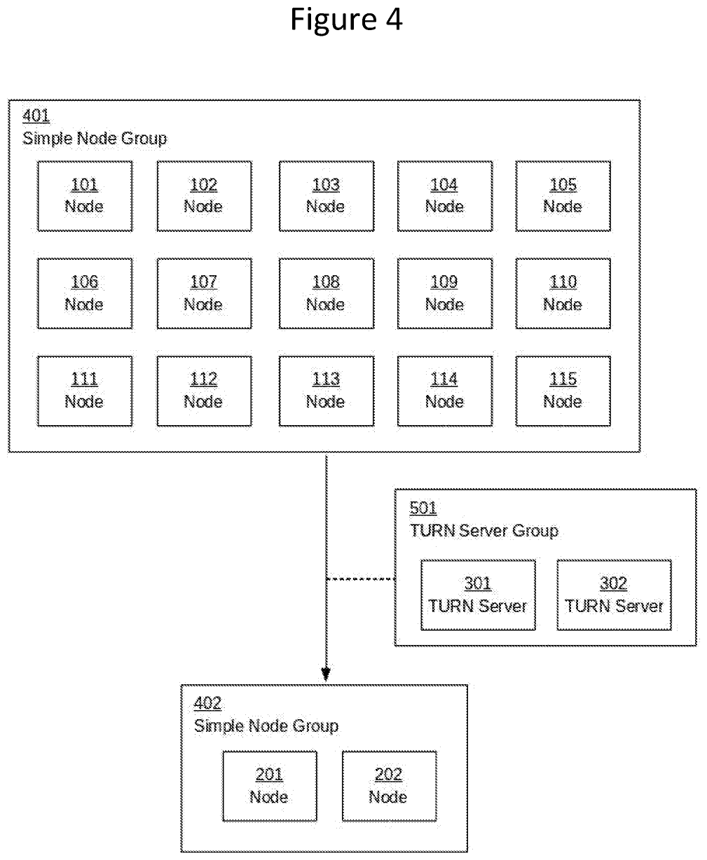

Network topologies may be devised and adjusted to align network goals and policies with the practicalities of heterogeneous hardware capabilities. This may be achieved, for example, by grouping similar machines together in distinct node groups. The node groups, including source, destination, simple, aggregation, distribution, and TURN server groups, for example, may serve as the building blocks for devising network deployments that are aligned with underlying physical network topologies. FIGS. 4 through 19 illustrate network arranges and streams for various media applications at various scales of deployment.

FIGS. 4 through 19 are intended to illustrate separate potential implementations of the concepts expressed herein. Item reference numbers used in a figure are generally, unless otherwise indicated, meant to refer to items in that figure only, and not to items in other deployment examples. The reuse of reference numbers is not intended to suggest that given entities are common to the various deployments. Rather, each deployment example is intended to stand on its own.

### EXAMPLE 1

### Broadcast Nodes and Observation Nodes with Remote Connectivity

FIG. 4 shows fifteen broadcast nodes, nodes 101 through 115, with local media capture devices (not shown), such as audio/video capture devices. Nodes 101-115 are configured by a controller (not shown) to belong to a node group 401. Observation nodes 201 and 202 are devices which belong to a node group 402. Observation nodes 201 and 202 may be running web browser sessions, for example. TURN servers 301 and 302 are assigned to a group 501, and are deployed to a data center with which all other nodes may initiate network connections (such as external-facing servers, or a public cloud provider.)

Node group 401 is linked to node group 402, and this link is associated with TURN server group 501. The TURN server group is sized according to redundancy requirements and the effective aggregation capacity for each TURN server, as determined by the number of simultaneous streams to be supported. The application may select the streams to send from 401 to 402 with either media propagation or direct media selection. This design enables observation nodes 201 and 202 to receive and display streams from the broadcast nodes 101-115.

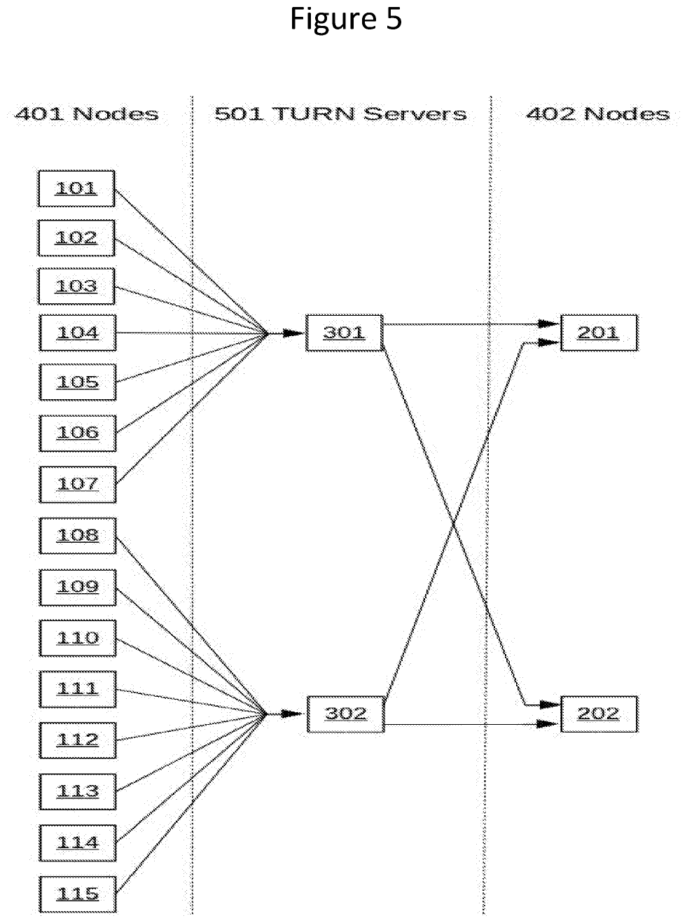

FIG. 5 shows the broadcast streams for the entities in FIG. 4, from the broadcast nodes 101-115 to the observation nodes 201 and 202. TURN server 301 aggregates seven streams from nodes 101-107 and relays these to nodes 201 and 202. TURN server 302 aggregates eight streams from nodes 108-115 and relays these to nodes 201 and 202. Both observation nodes 201 and 202 are connected to both TURN servers 301 and 302, and therefore receive all broadcast streams from nodes 101-115. In the example of FIG. 4, as reflected in FIG. 5, and the observation nodes 201 and 202 do not have network connections with the broadcast nodes 101-115. The media streams captured by the broadcast nodes 101-115 are relayed once, via one of the TURN servers 301 or 302.

### EXAMPLE 2

### Broadcast Nodes on a Shared Network and Remote Observation Nodes

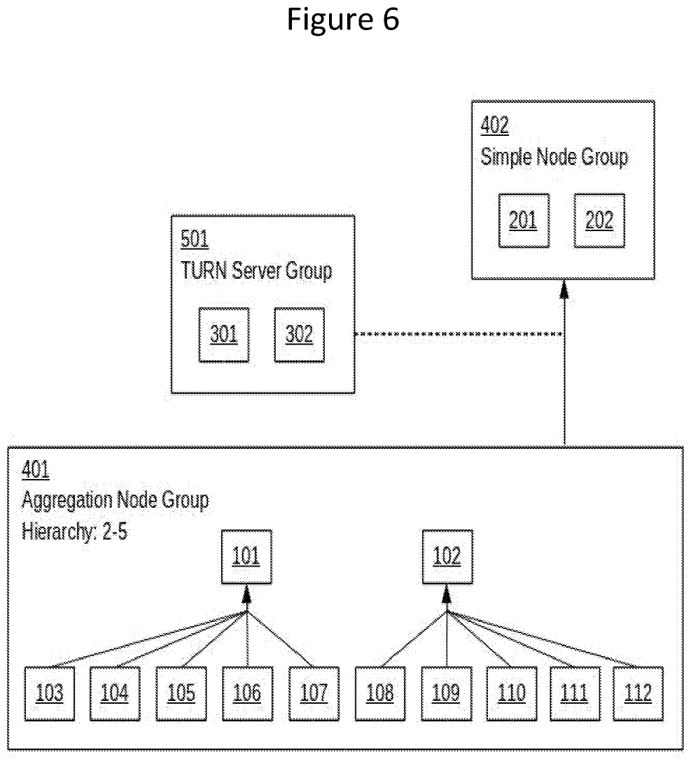

FIG. 6 illustrates an example of groups for broadcast nodes on a shared network and remote observation nodes. When broadcast nodes are deployed to a shared network such that each may make a direct peer connection with any other, the simple node group of the example of FIG. 4 may be replaced with an aggregation node group, which entails different resource usage and may enable deployments with higher capacity.

FIG. 6 shows twelve broadcast nodes 101 through 112, with local media capture devices (not shown.) Nodes 101-112 are configured by a controller (not shown) to belong to node group 401. Node group 401 is configured with an aggregation hierarchy of “2-5,” wherein the controller has selected the two nodes, nodes 101 and 102, for the top level. The controller has further selected five nodes, nodes 103-107 to relay to 101, and five nodes, nodes 108-112, to relay to node 102. Observation nodes 201 and 202 are entities such as web browser sessions, which belong to node group 402. TURN servers 301 and 302 are assigned to group 501, and are deployed to a data center with which all other nodes may initiate network connections, such as external-facing servers or a public cloud provider. The observation nodes 201 and 202 may be further deployed to networks other than the shared network of node group 401. Node group 401 is linked to node group 402, and this link is associated with TURN server group 501. An application may select the streams to send from 401 to 402 with either media propagation or direct media selection. This enables observation nodes 201 and 202 to receive and display streams from the broadcast nodes 101-112.

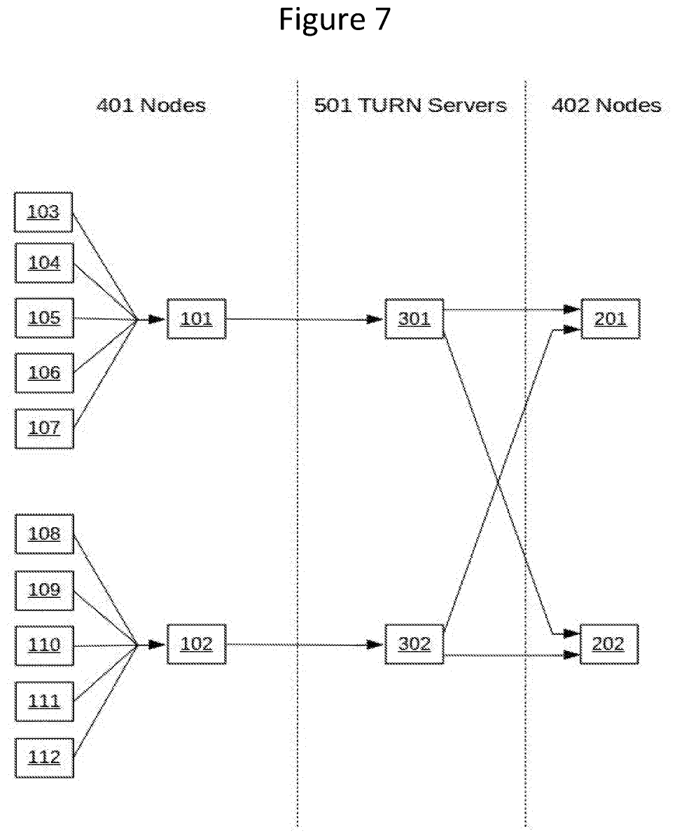

FIG. 7 shows the broadcast streams of the devices of FIG. 6 from the 103-112 nodes to the observation nodes 201 and 202. Nodes 103-107 capture media, such as audio/video, and stream to node 101. Nodes 108-112 capture media and stream to node 102. Nodes 101 and 102 capture these as inbound streams, and send all their inbound streams to both observation nodes 201 and 202.

In the example of FIG. 7, the TURN servers do not aggregate streams. Instead TURN server 301 receives inbound streams only from its connection with node 101, and TURN server 302 receives inbound streams only from its connection with 102. Each observation node 201 and 202 receives its streams from both TURN servers 301 and 302. The media streams captured by nodes 101 and 102 are relayed once, and the media captured by nodes 103-112 are relayed twice.

### EXAMPLE 3

### Broadcast Nodes and Observation Nodes on a Shared Network

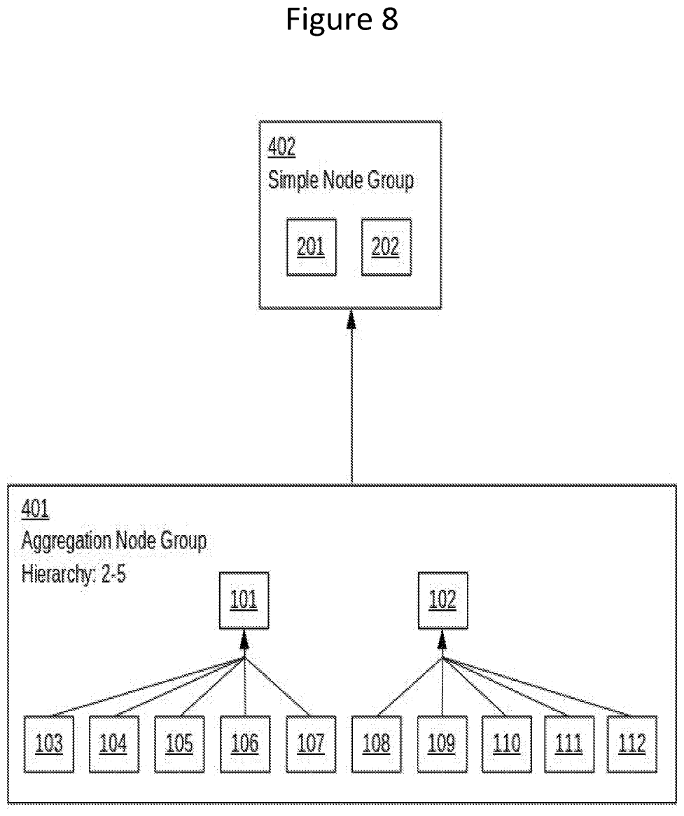

When broadcast and observation nodes are deployed to a shared network such that each may make a direct peer connection with any other node in the shared network, TURN servers are not required for connectivity. FIG. 8 shows twelve broadcast nodes, nodes 101 through 112, which have local media capture devices (not shown.) Nodes 101-112 are configured by a controller (not shown) to belong to node group 401. Node group 401 is configured with an aggregation hierarchy of 2-5, in which two nodes 101 and 102 are at the top level, and the five nodes 103-107 to relay to node 101, and the five nodes 108-112 to relay to node 102. Observation nodes 201 and 202 are entities such as web browser sessions, which belong to node group 402. Node group 401 is linked to node group 402, and this link is not associated a TURN server group. An application may select the streams to send from 401 to 402 with either media propagation or direct media selection. This design enables observation nodes 201 and 202 to receive and display streams from the broadcast nodes 101-112 across a shared network.

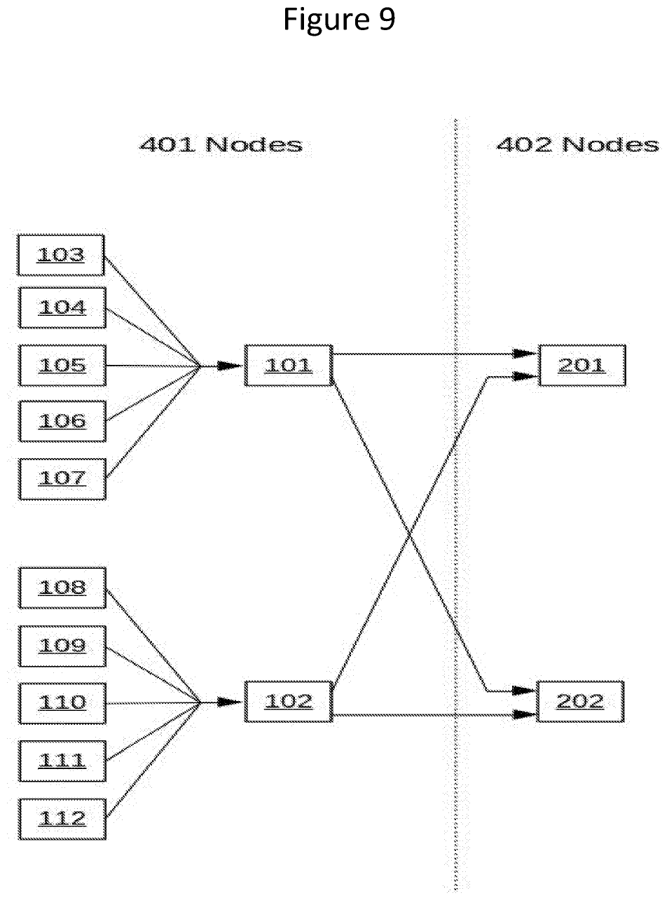

FIG. 9 shows the broadcast streams of FIG. 8 from the broadcast nodes 101-112 to the observation nodes 201 and 202. Nodes 103-107 capture media and stream to node 101, and nodes 108-112 capture media and stream to node 102. Nodes 101 and 102 capture media and send all inbound streams to both observation nodes 201 and 202. The media captured by nodes 101 and 102 is sent directly to the observation nodes 201 and 202 without relaying. The media captured by nodes 103-112 are relayed once.

### EXAMPLE 4

### Wide-Distribution Surveillance Application on a Shared Network

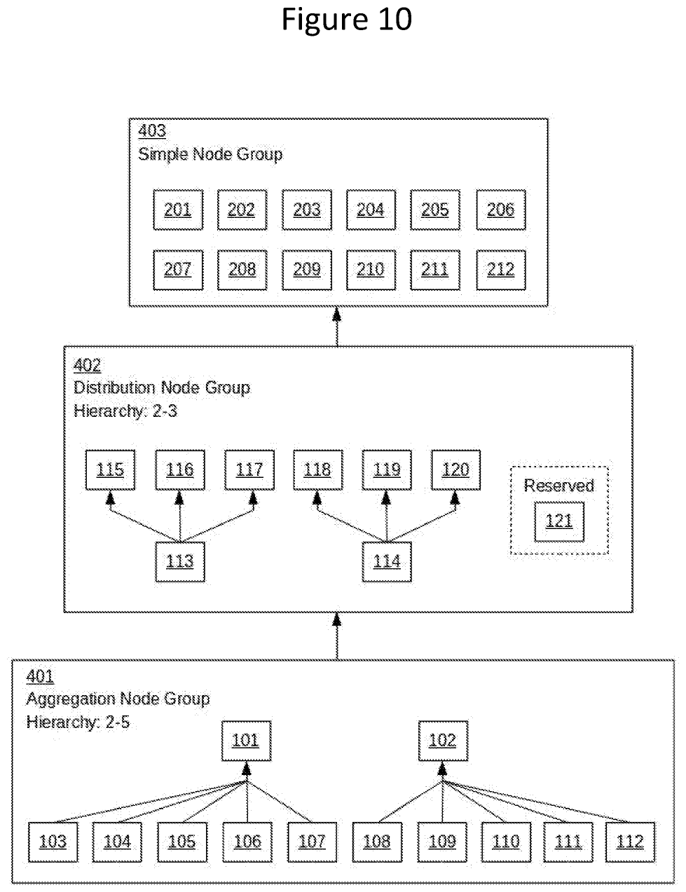

The addition of a distribution node group increases the scale of observation nodes that an implementation may support. FIG. 10 shows twelve broadcast nodes, nodes 101 through 112, with local media capture devices (not shown.) Nodes 101-112 are configured with a controller (not shown) to belong to a node group 401. Node group 401 is configured with an aggregation hierarchy of 2-5, in which the two nodes 101 and 102 are at the top level, the five nodes 103-107 to relay to node 101, and the five nodes 108-112 to relay to node 102.

The nine nodes 115-121 are dedicated relay devices, such as servers or virtual machines. These nodes 115-121 do not use media capture devices, and are configured with the controller to belong to node group 402. Node group 402 is configured with a distribution hierarchy of 2-3, in which the two nodes 113 and 114 are the bottom level. Three nodes 115-117 receive streams from node 113, the three nodes 118-120 receive streams from node 114. Node 121 is held in reserve.

Twelve observation nodes 201-212 are entities such as web browser sessions which belong to a node group 403. Node group 401 is linked to node group 402, and node group 402 is linked to node group 403. Neither link is associated with a TURN server group. The link from group 401 to group 402 is configured for media propagation of all media sources. The application may select the streams to send from 402 to 403 with either media propagation or direct media selection. This design enables observation nodes 201-212 to receive and display streams from the broadcast nodes 101-112 across a shared network.

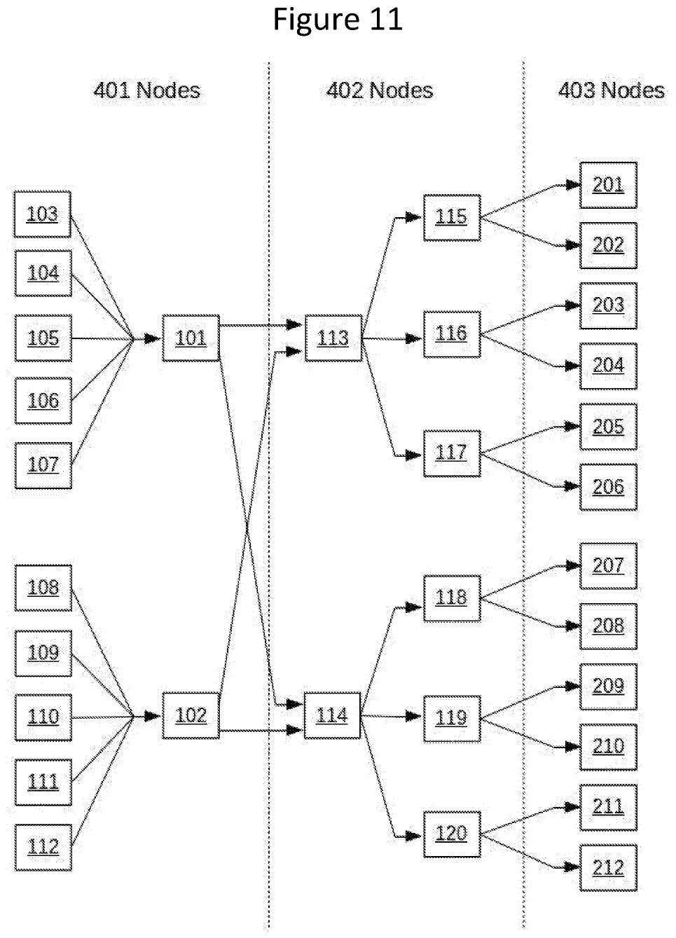

FIG. 11 shows the broadcast streams of FIG. 10 from all broadcast nodes to all observation nodes. Nodes 103-107 capture media and stream to node 101, and nodes 108-112 capture media and stream to node 102. Nodes 101 and 102 capture media and send all inbound streams to both distribution nodes 113 and 114. Distribution node 113 sends its inbound streams to the three nodes 115-117. Distribution node 114 sends its inbound streams to the three nodes 118-120. Distribution node 115 sends its inbound streams to the two observation nodes 201 and 202. Likewise each distribution node 116-120 sends its inbound streams to two observation nodes, distributed evenly across nodes 203-212. The media captured by nodes 101 and 102 are relayed twice, and the media captured by nodes 103-112 are relayed three times.

### EXAMPLE 5

### Large-Scale Surveillance Application

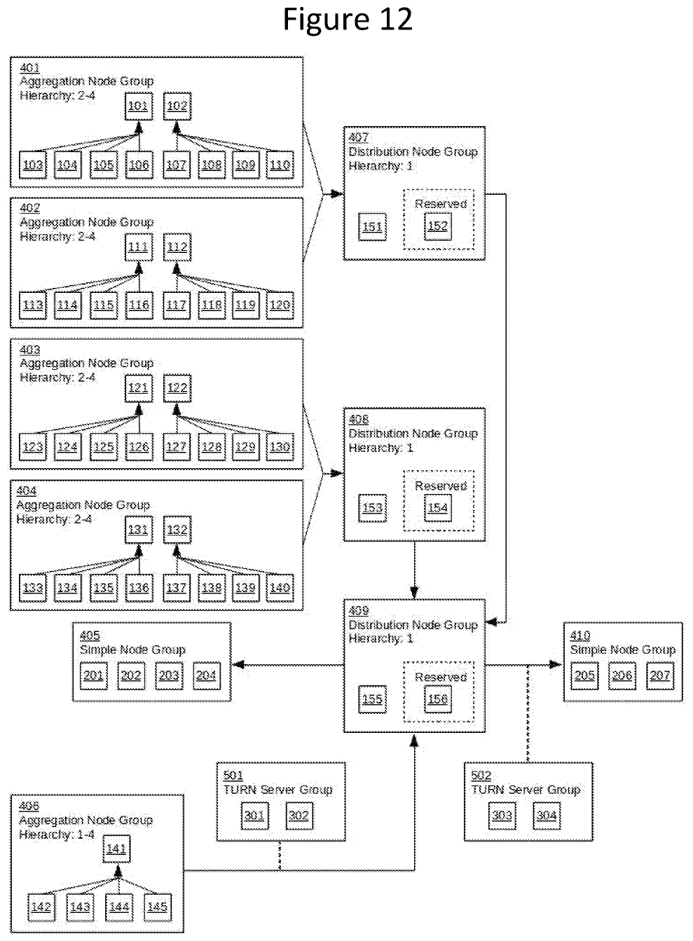

FIG. 12 shows an example of a larger-scale configuration for a surveillance application that is aligned with an underlying network topology. Each aggregation node group 401-404 corresponds to set of broadcast nodes on a shared network such as a LAN or VLAN, which is configured on a network switch, for example, per floor of a building in a campus network.

Simple node group 405 corresponds to observation nodes deployed across the same campus network. Aggregation node group 406 corresponds to broadcast nodes on a shared network in a remote office. Distribution node groups 407-409 correspond to relay devices deployed on the campus network. Simple node group 410 corresponds to remote observation nodes. TURN server groups 501 and 502 are deployed to a data center with which all other nodes may initiate network connections, such as external-facing servers on the campus network or a public cloud provider.

The ten broadcast nodes 101-110 belong to node group 401, which is configured with an aggregation hierarchy of 2-4. This configuration selects the two nodes 101 and 102 for the top level, the four nodes 103-106 to relay to node 101, and the four nodes 107-110 to relay to node 102. The broadcast nodes 111-140 and node groups 402-404 are likewise configured. The broadcast nodes 141-145 belong to node group 406, which is configured with an aggregation hierarchy of 1-4. This configuration selects node 141 for the top level and the four nodes 142-145 to relay to 141.

Distribution nodes with different computational resources may be apportioned into separate groups such that each group has members with similar capabilities, and such that those with fewer resources relay fewer streams. The example of FIG. 12 apportions nodes with higher capabilities to the central node group 409. The two nodes 151 and 152 are dedicated relay devices belonging to node group 407, which has a distribution hierarchy of 1. This configuration selects node 151 for distribution and node 152 as a backup device reserved on standby. The distribution nodes 153-156 and node groups 408 and 409 are likewise configured. Node groups 401 and 402 are linked to 407 with media propagation. Node groups 403 and 404 are linked to 408 with media propagation. Node groups 407 and 408 are linked to 409 with media propagation. Node group 406 is linked to 409 with media propagation, and this link is assigned to TURN server group 501 comprising TURN servers 301 and 302.

The four observation nodes 201-204 belong to simple node group 405. The three remote observation nodes 205-207 belong to simple node group 510. Node group 409 is linked to 405 without media propagation. Node group 409 is separately linked to 410, also without media propagation, and this link is assigned to TURN server group 502 comprising TURN servers 303 and 304.

This design enables observation nodes 201-207 to receive and display streams from the broadcast nodes 101-145. The media captured by all broadcast nodes 101-145 is propagated to distribution node 155 in node group 409, and the application relays broadcast streams from node group 409 to the observation nodes 201-207 with direct media selection.

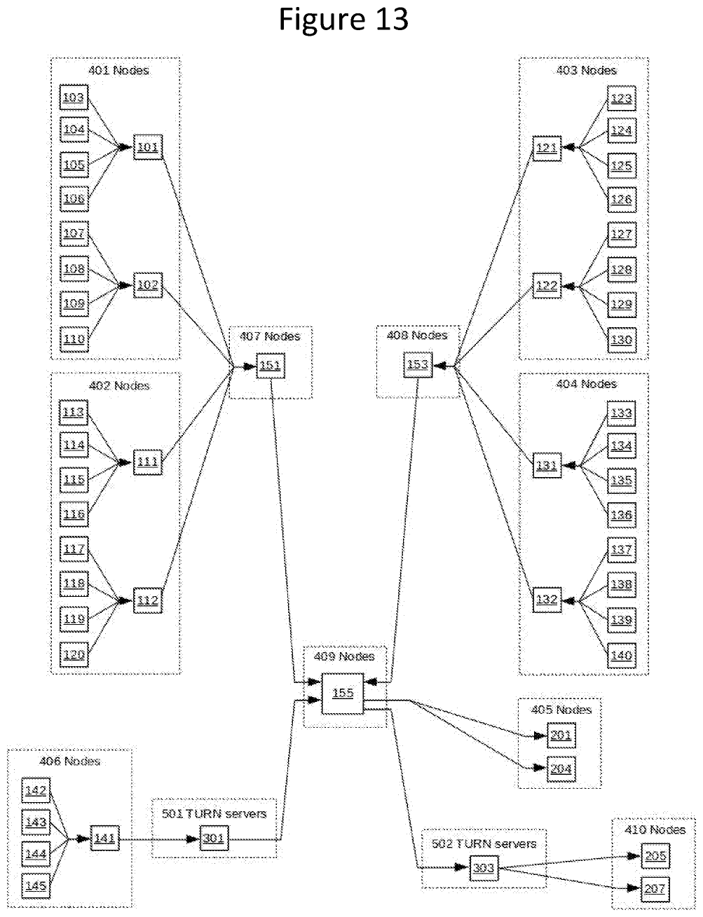

FIG. 13 shows streams for the example of FIG. 12. This includes broadcast streams for direct media selection from distribution node 155 to four observation nodes. Nodes 103-106 capture media and stream to node 101, and nodes 107-110 capture media and stream to node 102. Nodes 101 and 102 capture media and send all inbound streams to distribution node 151. Nodes 113-116 capture and stream to node 111. Nodes 117-120 capture and stream to 112. Nodes 111 and 112 capture and relay streams to 151. Nodes 123-126 capture and stream to 121. Nodes 127-130 capture and stream to node 122. Nodes 133-136 capture and stream to 131. Nodes 137-140 capture and stream to node 132. Nodes 121, 122, 131 and 132 capture and relay streams to node 153. Distribution nodes 151 and 153 relay all inbound streams to node 155. Remote broadcast nodes 142-145 capture and stream to 141. 141 captures and relays streams to 155 via TURN server 301.

Distribution node 155 sends streams per direct media selection directly to local observation nodes 201 and 204, and indirectly to remote observation nodes 205 and 207 via TURN server 303. The media captured by broadcast nodes 101, 102, 111, 112, 121, 122, 131, 132, 141 are relayed twice to local observation nodes 201 and 204, and are relayed three times to remote observation nodes 205 and 207. All other broadcast nodes have one additional relay in the streaming path to observation nodes.

### EXAMPLE 6

### Response to Failures in a Large-Scale Surveillance Application

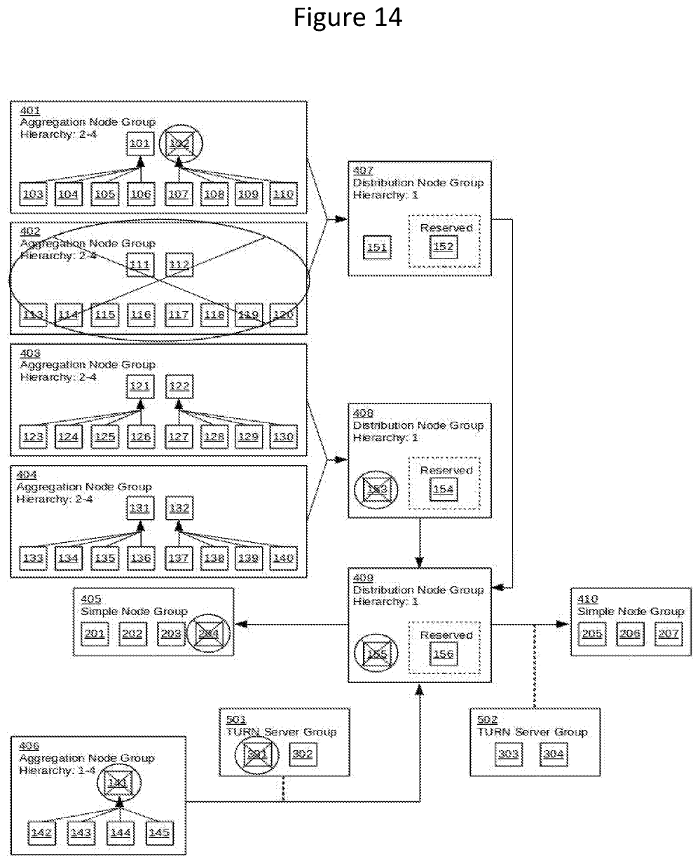

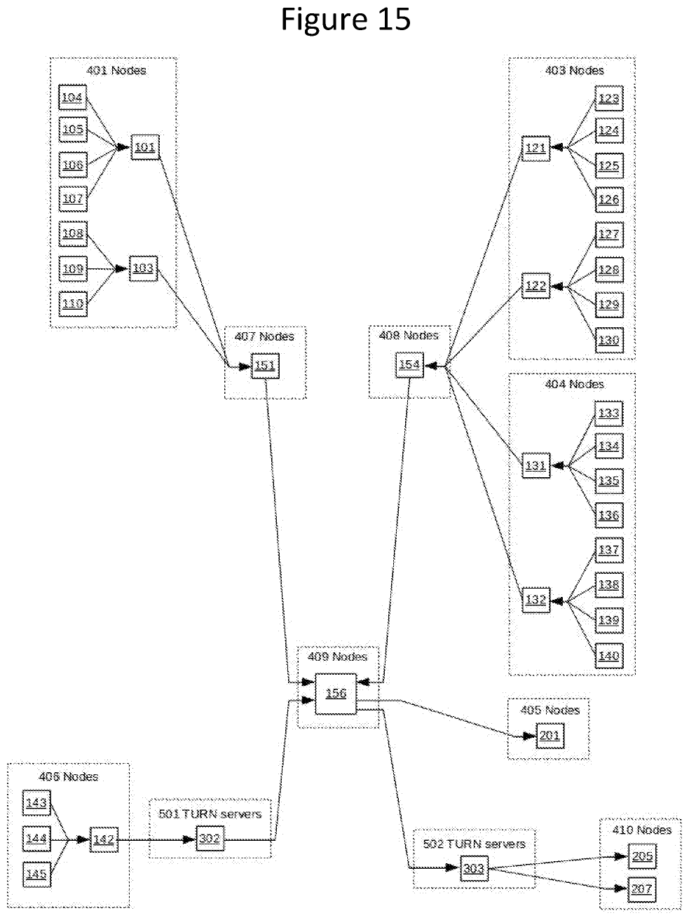

FIG. 14 shows an example of large-scale surveillance application network of FIG. 12 as it experiences failures and responds thereto. In the example of FIG. 14, the location of broadcast nodes 111-120 represented by node group 402 becomes unavailable, e.g., as may occur from an extended power outage. Individual node failures also occur for broadcast nodes 102 and 141, distribution nodes 153 and 155, observation node 204, and TURN server 301. FIG. 15 shows the broadcast streams updated in response to these failures.

### EXAMPLE 7

### Extended Large-Scale Surveillance Application

Deployment configurations, such as those in the examples of FIGS. 12 and 14, may be extended to support additional broadcast and observation nodes. Instead of using nodes with greater computational resources to relay a greater number of streams over peer connections, additional nodes of lower capabilities may be deployed to larger node groups.

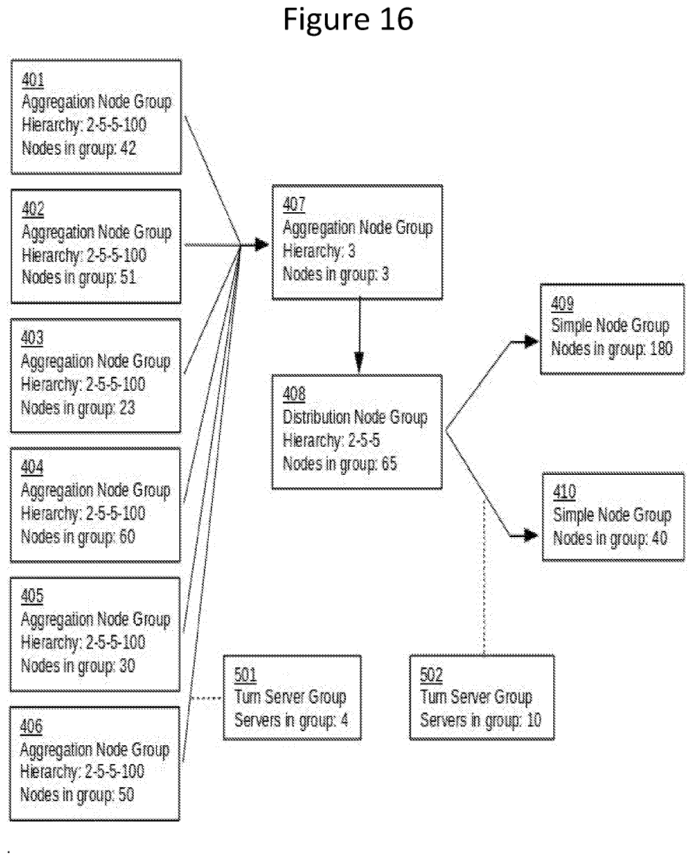

FIG. 16 shows an extended deployment with larger aggregation and distribution node groups to support a greater number of broadcast and observation nodes. Aggregation node groups 401-406 comprise broadcast nodes deployed to shared networks. Node groups 406 and 407 comprise dedicated relay nodes deployed to a central data center. The link from 406 to 407 is associated with TURN server group 501 to provide remote access across a public network. Node group 409 comprises observation nodes deployed to a protected private network, and node group 410 comprises remote observation nodes connected across a public network. The link from 408 to 410 is associated with TURN server group 502 to provide remote access across a public network.

### EXAMPLE 8

### General Conference Application

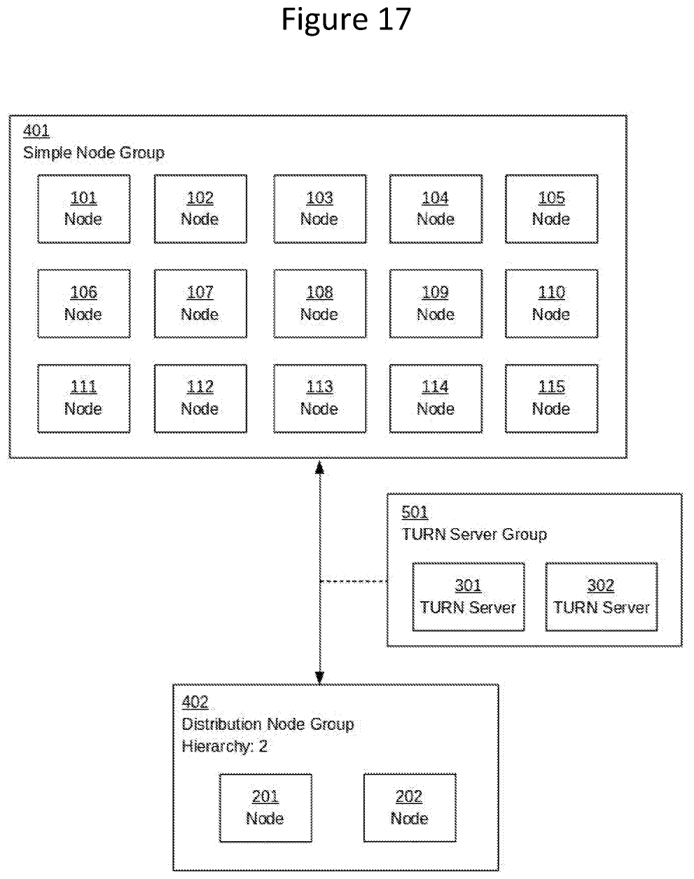

Two-way communication may be implemented with the previous examples by specifying direct media selection from an observation node to a broadcast node. These direct media streams are relayed by the connecting node groups. An application requiring infrastructure support for general conferencing may be implemented with a design such as shown in FIG. 17. In FIG. 17, nodes 101-115 are assigned to a simple node group 401. Distribution nodes 201 and 202 are deployed to dedicated relay devices and assigned to a node group 402. TURN servers 301 and 302 are deployed to a data center with which all other nodes may initiate network connections, such as external-facing servers or a public cloud provider. Node group 401 is linked to group 402, and this link is associated with a TURN server group 501. This design enables nodes 101-115 to participate in an audio/video conference.

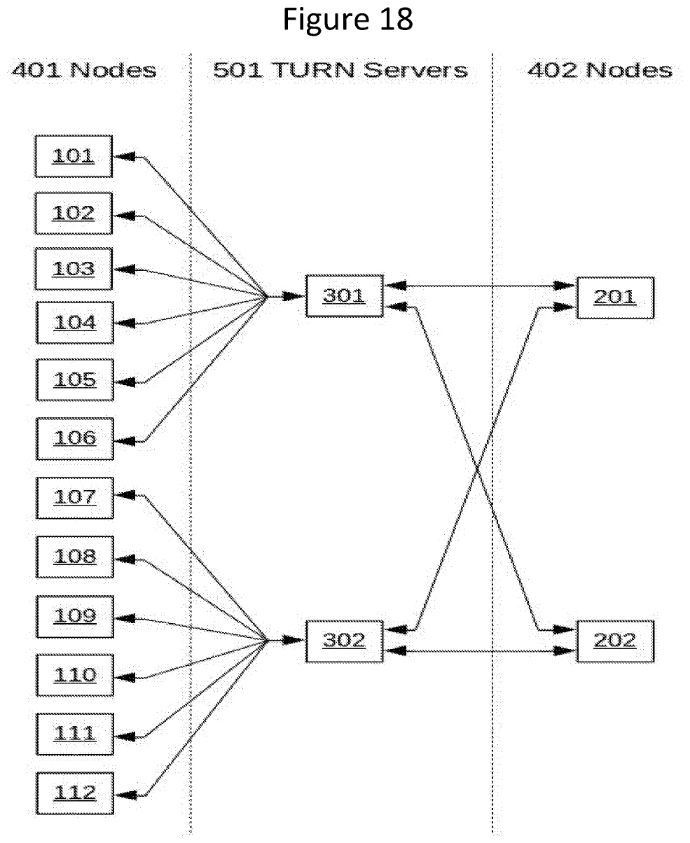

FIG. 18 shows these conference streams among nodes 101-112. Nodes 101-106 capture media and stream to both distribution nodes 201 and 202 via TURN server 301. Nodes 107-112 capture media and stream to both distribution nodes 201 and 202 via TURN server. Nodes 101-112 receive streams from all other participating nodes via both TURN servers.

### EXAMPLE 9

### Large-Scale Conference Application

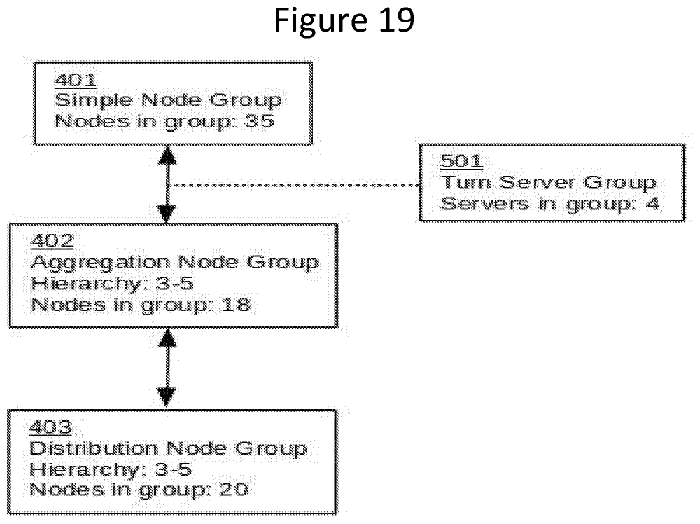

FIG. 19 illustrates the use of groups for a large-scale conferencing application that is implemented with corresponding provisioned aggregation and distribution node groups. FIG. 19 shows a node group 401 comprising 35 participant nodes. Node group 401 is linked a to node group 402. Node group 402 comprises 18 dedicated relay nodes configured for aggregation. The link between node group 401 and node group 402 is associated with a TURN server group 501 to provide remote access between the remote participant nodes and the dedicate aggregation nodes. Node group 402 is linked to a distribution node group 403 comprising 20 dedicated distribution nodes. The aggregation and distribution nodes are deployed to a private network with direct interconnectivity. This design enables all members of node group 401 to participate in an audio/video conference.

We claim:

1. A first apparatus, comprising a processor, a memory, and communication circuitry, the first apparatus being connected to a communications network via its communication circuitry, the first apparatus further comprising computer-executable instructions stored in the memory of the first apparatus which, when executed by the processor of the first apparatus, cause the first apparatus to:
receive, from a second apparatus via a remote connection, information regarding a first media stream, the second apparatus being a network controller, wherein the information regarding the first media stream comprises an identification of a source node for the first media stream and a sink node for the first media stream;
determine that the first media stream is available to the first apparatus;
select, based on the information regarding the first media stream, a third apparatus to receive the first media stream;
determine that the third apparatus is available to receive the first media stream;
establish, based on the determinations that the first media stream and the third apparatus are available, a peer connection with the third apparatus; and
stream, to the third apparatus via the peer connection, the first media stream.

2. The first apparatus of claim 1, wherein, the instructions further cause the first apparatus to signal, to the second apparatus, a state of a peer connection the first apparatus.

3. The first apparatus of claim 2, wherein, the state of the peer connection of the first apparatus is selected from a list comprising on, off, turning on, and turning off.

4. The first apparatus of claim 3, wherein, the instructions further cause the first apparatus to signal, to the second apparatus, a state of the first stream.

5. The first apparatus of claim 1, wherein:
the first apparatus has a local connection to a media capture device, the media capture device providing the first media stream;
the source node for the first media stream is the first apparatus;
the instructions further cause the first apparatus to label the first media stream with an identifier of the first apparatus.

6. The first apparatus of claim 1, wherein, the instructions further cause the first apparatus to:
receive the first media stream in the form of two or more component streams; and
combine the component streams into a single stream for transmission to the third apparatus.

7. The first apparatus of claim 6, wherein, the two component streams are video and audio streams of a monitored patient.

8. The first apparatus of claim 1, wherein, the instructions further cause the first apparatus to receive, from the second apparatus, a configuration, the configuration pertaining to acting as an aggregation node in an aggregation group.

9. The first apparatus of claim 8, wherein:
the configuration comprises an indication of a node higher in an aggregation group hierarchy, and
the instructions further cause the first apparatus to forward any active media streams to the node higher in the aggregation group hierarchy.

10. The first apparatus of claim 1, wherein, the instructions further cause the first apparatus to receive, from the second apparatus, a configuration, the configuration pertaining to acting as a distribution node in a distribution group.

11. The first apparatus of claim 10, wherein:
the configuration comprises an indication of a set of nodes lower in a distribution group hierarchy, and
the instructions further cause the first apparatus to forward any active media streams to the set of nodes lower in the distribution group hierarchy.

12. A second apparatus comprising a processor, a memory, and communication circuitry, the second apparatus being connected to a communications network via its communication circuitry, the second apparatus further comprising computer-executable instructions stored in the memory of the second apparatus which, when executed by the processor of the first second apparatus, cause the second apparatus to:
maintain a map, the map comprising data regarding a plurality of media stream devices, a plurality of media streams, wherein the media devices comprise media stream sink devices, media stream source devices, and media stream relay devices, the map further comprising a set of preferred routes for the media streams, wherein each route comprises one or more pair, each pair comprising a media source device and a media sink device;
transmit, to each media stream device, a portion of the map, the portion of the map pertaining to the media stream device;
receive, from one more of the media stream devices, status data;
revise, based on the status data, the map.

13. The second apparatus of claim 12, wherein, the status data comprises a peer connection status selected from a list comprising on, off, turning on, and turning off.

14. The second apparatus of claim 12, wherein, a portion of the map for a first apparatus comprises an indication that the first apparatus is to source a first media stream from a media capture device on a local connection to the first apparatus.

15. The second apparatus of claim 12, wherein, a portion of the map for a first apparatus comprises an indication that the first apparatus is to forward any active streams of the first apparatus to a higher node in an aggregation node hierarchy.

16. The second apparatus of claim 12, wherein, a portion of the map for a first apparatus comprises an indication that the first apparatus is to forward any active streams of the first apparatus to a number of lower node in a distribution node hierarchy.

17. The second apparatus of claim 12, wherein the second apparatus comprises a cluster of servers.

18. A system, comprising:
a controller and a plurality of media stream devices, wherein the media stream devices comprise media stream sink devices, media stream source devices, and media stream relay devices,
wherein the controller is arranged to:
maintain a map, the map comprising data regarding the plurality of media stream devices and a plurality of media streams, the map further comprising a set of preferred routes for the media streams, wherein each route comprises one or more pairs, each pair comprising a media source device and a media sink device;
transmit, to each media stream device, a portion of the map, the portion of the map pertaining to the media stream device;
receive, from one more of the media stream devices, status data; and
revise, based on the status data, the map; and
wherein each media stream device is arranged to:
initiate a peer connection with a second media stream device, in accordance with the portion of the map pertaining to the media stream device, upon determination that a first media stream and the second media stream device are available; and
stream the first media stream to the other media stream device.

19. The system of claim 18, wherein, the controller neither receives nor sends any media stream.

20. The system of claim 19, wherein the controller is a cluster of servers.

21. The system of claim 18, wherein the media stream relay devices comprise TURN servers.

22. The system of claim 18, wherein, the media stream relay devices comprise a distribution node group.

23. The system of claim 18, wherein, the media stream relay devices comprise an aggregation node group.

24. The system of claim 18, wherein:
the media stream source devices comprise patient monitoring devices providing video and audio streams of monitored patients; and
the media stream sink devices comprise patient observer devices providing observers with access to the video and audio streams of monitored patients.
